SEQUENCE LISTING (13 sequences)

SEQ ID NO: 1 - DNA, 11 bp, Unknown
  EcoNI restriction enzyme recognition site
cctnnnnnagg

SEQ ID NO: 2 - DNA, 11 bp, Unknown
  Eam1105I restricition enzyme recognition site
gacnnnnngtc

SEQ ID NO: 3 - DNA, 15 bp, Unknown
  XcmI restriction enzyme recognition site
ccannnnnnnnntgg

SEQ ID NO: 4 - DNA, 12 bp, Unknown
  DrdI restriction enzyme recognition site
gacnnnnnngtc

SEQ ID NO: 5 - DNA, 17 bp, Unknown
  HauII restriction enzyme recognition site
tggccannnnnnnnnnn

SEQ ID NO: 6 - DNA, 11 bp, Unknown
  BglI restriction enzyme recognition site
gccnnnnnggc

SEQ ID NO: 7 - DNA, 11 bp, Unknown
  BsiYI restriction enzyme recognition site
ccnnnnnnngg

SEQ ID NO: 8 - DNA, 11 bp, Unknown
  BstAPI restriction enzyme recognition site
gcannnnntgc

SEQ ID NO: 9 - DNA, 11 bp, Unknown
  MwoI restriction enzyme recognition site
gcnnnnnnngc

SEQ ID NO: 10 - DNA, 11 bp, Unknown
  PflMI restriction enzyme recognition site
ccannnnntgg

SEQ ID NO: 11 - DNA, 13 bp, Unknown
  SfiI restriction enzyme recognition site
ggccnnnnnggcc

SEQ ID NO: 12 - DNA, 12 bp, Unknown
  BstXI restriction enzyme recognition site
ccannnnnntgg

SEQ ID NO: 13 - DNA, 11 bp, Unknown
  ApaBI restriction enzyme recognition site
gcannnnntgc

(19) United States
(12) Patent Application Publication
(10) Pub. No.: US 2014/0038240 A1
(43) Pub. Date: Feb. 6, 2014

(54) METHODS FOR MULTIPART, MODULAR AND SCARLESS ASSEMBLY OF DNA MOLECULES

(71) Applicants: Karsten Temme, San Mateo, CA (US); Alvin Tamsir, San Mateo, CA (US)

(72) Inventors: Karsten Temme, San Mateo, CA (US); Alvin Tamsir, San Mateo, CA (US)

(73) Assignee: PIVOT BIO, INC., San Francisco, CA (US)

(21) Appl. No.: 13/939,110

(22) Filed: Jul. 10, 2013

Related U.S. Application Data
(60) Provisional application No. 61/670,061, filed on Jul. 10, 2012.
(60) Provisional application No. 61/789,032, filed on Mar. 15, 2013.

(51) Int. Cl. C12P 19/34

(52) U.S. Cl. CPC C12P 19/34
USPC 435/91.53; 435/196

(57) ABSTRACT

The present invention consists of methods for joining DNA molecules (parts) together to form larger DNA molecules (assemblies) of specified sequence and organization. The invention exhibits three necessary characteristics. Firstly, the invention enables 2 or more parts to be joined in a single reaction. Secondly, the seam between joined parts is scarless, producing no residual sequence dependencies like restriction enzyme recognition sites. Thirdly, parts are modular and can easily be reused in novel assemblies without modification. Prior technologies have exhibited no more than two of the three necessary characteristics, limiting their utility in synthesizing and editing DNA molecules of arbitrary sequence.

### PRIORITY

The present application claims priority to, and the benefit of, U.S. Provisional Application No. 61/670,061 filed Jul. 10, 2012, and U.S. Provisional Application No. 61/789,032 filed Mar. 15, 2013, each of which is hereby incorporated by reference in its entirety.

### FIELD OF THE INVENTION

The present invention relates to methods for multipart, modular and scarless assembly of nucleic acids, including for high-throughput, automated, and/or large scale engineering of biological systems.

### BACKGROUND

A key concept within synthetic biology is that biological DNA parts can be standardized, abstracted, and combined to produce complex, engineered systems. Parts are routinely generated via cloning from the DNA of organisms or using DNA synthesis. However, assembling parts into complex systems remains a key bottleneck in the synthetic biology workflow.

Numerous technologies have been developed to facilitate DNA assembly, yet none provide a robust solution. (See, e.g., U.S. Patent Application No. 2010/0035768, U.S. Patent Application No. US 2012/0040870; Engler C. et al., A One Pot, One Step, Precision Cloning Method with High Throughput Capability, PLoS ONE 3(11):e3647 (2008), doi:10.1371/journal.pone.0003647; Weber E., et al., A Modular Cloning System for Standardized Assembly of Multigene Constructs. PLoS ONE 6(2):e16765 (2011), doi:10.1371/journal.pone.0016765; and Ellis, T., et al. DNA assembly for synthetic biology: from parts to pathways and beyond, Integr. Biol., 3:109-118 (2011), DOI: 10.1039/C01B00070A 2; and information found on the World Wide Web at j5.jbei.org/j5manual/pages/1.html; all of which are incorporated by reference herein in their entireties.)

There remains a need in the synthetic biology field by which biological DNA parts can be routinely combined, and at high-throughput, to produce complex, engineered systems. The present invention meets these objectives.

### SUMMARY OF THE INVENTION

The present invention provides for multipart, modular and scarless nucleic acid assembly in vitro. In some embodiments, the DNA assembly reactions, which can proceed in parallel and series, are designed computationally based on a desired sequence. For example, the nucleic acid assembly may involve a plurality of reactions in parallel and/or in series that are designed in silico for accurate, cost-effective engineering of biological systems. In some embodiments the methods and kits described herein can be employed with high-throughput, automated processing systems.

In some embodiments, the invention provides a method for constructing a scarless nucleic acid molecule comprising a plurality of heterologous parts. Nucleases and nucleic acid staples or adaptors are selected, as described herein, to assemble the heterologous parts into a scarless nucleic acid molecule. Nuclease and ligation reactions can take place in parallel and/or in series, as needed for optimum control of the process. The process can be controlled computationally by user inputs, with reaction assembly and processing taking place by automation.

In some embodiments, the method comprises generating a first nucleic acid molecule having a single stranded terminus, generating a second nucleic acid molecule having a single stranded terminus, and then ligating the first and second nucleic acid molecules with the aid of an intervening linker molecule such that the ligation product corresponds to the combined sequence of the first and second nucleic acid molecules. In some embodiments, the nucleic acid molecule is a DNA molecule. An algorithm can be employed to computationally determine, identify and/or optimize any of the parts, enzymes and/or other reagents to be employed with the present methods.

Scarless nucleic acid assembly according to the methods of the present invention requires two classes of enzymes. The first enzyme catalyzes the formation of short (about 1 bp to 8 bp), single stranded 5′-overhangs on a nucleic acid. The second enzyme catalyzes the formation of short (about 1 bp to 8 bp), single stranded 3′-overhangs on a nucleic acid. Each of these enzymes and overhang size can be independently selected, and can be a Type II restriction enzyme in some embodiments.

In some embodiments, the linker is a staple. A staple may be single stranded and can be DNA or RNA. In some embodiments, the staple is a defined sequence capable of binding with perfect complementarity to the single stranded DNA termini generated on the first and second DNA molecules. In some embodiments, the staple binds to a single stranded DNA terminus with a 3′-overhang on a first DNA molecule and a single stranded DNA terminus with a 5′-overhang on a second DNA molecule. In some embodiments, the staple binds to a single stranded DNA terminus with a 5′-overhang on a first DNA molecule and a single stranded DNA terminus with a 3′-overhang on a second DNA molecule.

In some aspects, the present invention provides a plurality of reaction mixtures for performing one and/or a series of reaction mixtures for scarless nucleic acid assembly.

### BRIEF DESCRIPTION OF THE DRAWINGS

FIG. 1: Schematic diagram showing Staple Implementation and Adapter Implementation for multipart, modular and scarless assembly (MMS).

FIG. 2: Assembly of two DNA parts using a “staple” linker. Two input DNA parts each with a size of 250 bp and 400 bp are ligated together to form a 650 bp product. Lane 1: 100 bp NEB DNA ladder. Lane 2: Input DNA only. Lane 3: Input DNA (without oligonucleotide “staple”) after ligation reaction. Lane 4: Input DNA+oligonucleotide staple after ligation reaction.

FIG. 3: Assembly of two DNA parts using an “adapter” linker. Lane 1: 1 kb NEB ladder. Lane 2: Two input DNA parts of sizes 1800 bp and 300 bp are assembled to form a 2100 bp product. Lane 3: Two input DNA parts of sizes 700 bp and 1800 bp are assembled to form a 2500 bp product.

FIG. 4: Isothermal Scarless Subcloning. Reaction mixture containing T4 ligase buffer, T7 DNA ligase, BsaI and BsaXI, and DNA parts. Isothermal reaction was performed at 37° C. for 1 hr. Colony PCRs and sequencing show 11 of 12 clones assembled correctly.

FIG. 5: Scarless Assembly of Multiple Parts. Reaction mixture containing T4 ligase buffer, T7 DNA ligase, BsaI and BstXI, and DNA parts. Isothermal reaction was performed at 37° C. for 8 hr. Colony PCRs and sequencing show 6 of 12 clones assembled correctly.

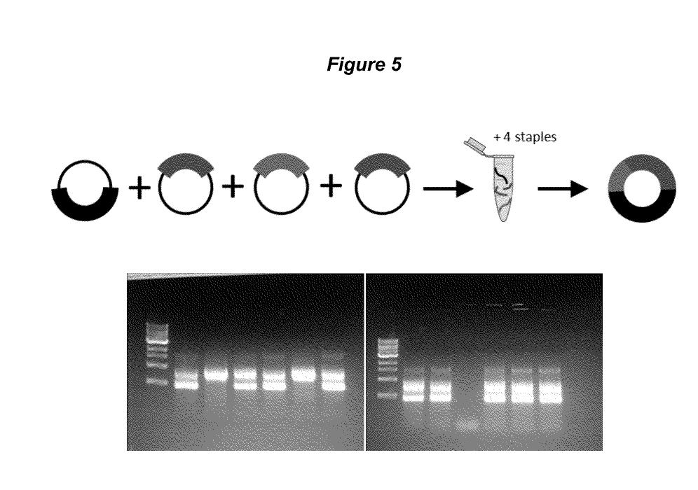

FIG. 6: Multiplex Assembly in One Tube. Reaction mixture containing T4 ligase buffer, T7 DNA ligase, BsaI and BstXI, and DNA parts. Isothermal reaction was performed at 37° C. for 8 hr. Colony PCRs and sequencing show 23 of 24 clones assembled correctly.

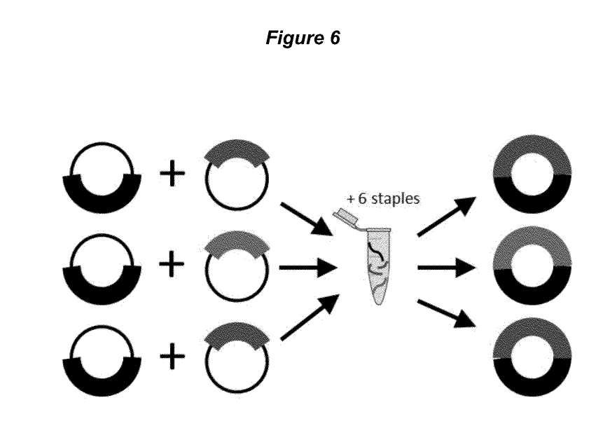

### DETAILED DESCRIPTION OF THE INVENTION

The present invention provides for multipart, modular and scarless nucleic acid assembly in vitro. In some embodiments, the DNA assembly reactions, which can proceed in parallel and series, are designed computationally based on a desired sequence. For example, the nucleic acid assembly may involve a plurality of reactions in parallel and/or in series that are designed in silico for accurate, cost-effective engineering of biological systems. In some embodiments the methods and kits described herein can be employed with high-throughput, automated processing systems.

The term “scarless” refers to the fact that no changes or undesired sequences are introduced into assembled DNA by the reactions. The combined sequence will correspond to the exact sequence desired with no changes being introduced by the restriction enzyme/ligation procedure. The combined sequence can correspond exactly to a natural sequence, an engineered sequence, a synthetic sequence or any other desired reference sequence.

The term “modular” refers to the fact that prepared nucleic acid parts can be ligated with any other prepared nucleic acid parts without dependencies on the nucleic acid sequence of the two parts.

The term “multipart” refers to the fact that two or more nucleic acid parts can be ligated in a single in vitro reaction.

The term “reagent” can include any component of a reaction described herein. Reagents can include but are not limited to buffers, enzymes (e.g., nucleases, ligases) and nucleic acids (e.g., parts, linkers, staples). Nucleic acid reagents can include one or more chemically modified bases, including for example but not limited to phosphorothioates, locked nucleic acids (LNAs), peptide nucleic acids (PNAs), 2′-0Me nucleotides, methylphosphonates or morpholinos, as well as any other modifications known in the art and that one of skill would find useful for the present methods.

Perfect or near perfect complementarity occurs when two nucleic acids regions of interest share about 100%, about 99%, about 98%, about 97%, about 96%, about 95%, about 94%, about 93%, about 92%, about 91%, about 90%, about 89%, about 88%, about 85%, about 80%, about 75%, or about 70% sequence identity, homology or complementarity to one another.

In some embodiments, the method provides for assembly of any desired nucleic acid molecule, including DNA or RNA, as well as modified DNA and RNA molecules (e.g., nucleic acids containing chemically modified bases, such as but not limited to phosphorothioates, locked nucleic acids (LNAs), peptide nucleic acids (PNAs), 2′-0Me nucleotides, methylphosphonates or morpholinos). In some embodiments, assembly is via high-throughput methods and in some embodiments, said high-throughput methods are automated. The resulting DNA molecules can be at least 1 kb in length, at least 10 kb in length, at least 100 kb in length, or over 500 kb in length, or over 1000 kb in length.

In some embodiments, the invention involves computational selection of the desired DNA parts, and/or desired reagents, as well as design of optimal parallel and/or series reactions for generating the desired DNA product.

In some embodiments, the invention provides a method for constructing a scarless nucleic acid molecule comprising 2 or more heterologous parts, such as 5 or more, 10 or more, 15 or more, 20 or more, 30 or more, 40 or more, 50 or more, or 100 or more heterologous parts. Nucleases and nucleic acid staples and/or adaptors are selected, as described herein, to assemble the heterologous parts into a scarless nucleic acid molecule by ligation. The restriction and ligation reactions can take place in parallel and/or in series, as needed for optimum control of the process. The process can be controlled computationally by user inputs, with reaction assembly and processing taking place by automation.

In some embodiments, the method comprises generating a first nucleic acid molecule having a single stranded terminus, generating a second nucleic acid molecule having a single stranded terminus, and then ligating the first and second nucleic acid molecules with the aid of an intervening linker molecule such that the ligation product corresponds to the combined sequence of the first and second nucleic acid molecules. In some embodiments, the nucleic acid molecule is a DNA molecule. In some embodiments, an algorithm can be employed to computationally determine, identify and/or optimize any of the parts, enzymes and/or other reagents to be employed with the present methods. Ligation methods are well known in the art and any of these known ligation methods can be employed with the present invention.

In some embodiments, the first nucleic acid molecule or the second nucleic acid molecule have single stranded termini generated with a restriction enzyme. In some embodiments, the nucleic acid molecule is a DNA molecule.

Scarless nucleic acid assembly according to the methods of the present invention requires two classes of enzymes. The first enzyme catalyzes the formation of short (about 1 bp to 8 bp), single stranded 5′-overhangs on a nucleic acid. The second enzyme catalyzes the formation of short (about 1 bp to 8 bp), single stranded 3′-overhangs on a nucleic acid. Each of these enzymes and overhang size can be independently selected. In some embodiments, such restriction enzymes are selected from Type IIs, Type IIb, or Type IIp family enzymes. In some embodiments, in part in order to bypass constraints on nucleic acid sequences, the enzymes are selected from types that cleave the nucleic acid sequence at a position distal (about 1 bp to 25 bp) to the recognition site.

In some embodiments, the single stranded termini can include 5′-overhangs, 3′-overhangs which are independently selected. In some embodiments, the overhangs are independently selected from the following ranges: about 1 bp to 8 bp, about 2 bp to 8 bp, about 2 bp to 6 bp, about 3 bp to 6 bp, about 3 bp to 5 bp, about 2 bp to 6 bp, about 2 bp to 5 bp, about 1 bp to 5 bp, about 2 bp to 4 bp, about 1 bp to about 4 bp, about 1 bp to 3 bp or about 1 bp to 2 bp. In some embodiments, the overhangs are about 1 bp, 2 bp, 3 bp, 4 bp, 5 bp, 6 bp, 7 bp, or 8 bp or more in length.

In some embodiments, the restriction enzyme is a Type IIs restriction enzyme. The Type II restriction enzymes that find use with the methods of the present invention can generate a single stranded nucleic acid terminus with a 3′-overhang or a 5′-overhang. Enzyme properties can also be found on the World Wide Web at rebase.neb.com.

TABLE 1
Type II restrictions enzymes producing
5′-overhangs

| Length | Overhang | Enzymes | Recognition Sequence |
|---|---|---|---|
| 1 | N | BccI | CCATC (4/5) |
| 1 | N | BcefI | ACGGC (12/13) |
| 1 | N | BinI | GGATC (4/5) |
| 1 | N | EcoNI | CCTNN↓NNNAGG |
| 1 | N | Fnu4HI | GC↓NGC |
| 1 | N | PleI | GAGTC (4/5) |
| 1 | N | ScrFI | CC↓NGG |
| 1 | N | Tth111I | GACN↓NNGTC |
| 1 | S | CauII | CC↓SGG |
| 1 | W | BstNI | CC↓WGG |
| 2 | AT | Asi256I | G↓ATC |
| 2 | AT | CviAII | C↓ATG |
| 2 | CG | AciI | CCGC (−3/−1) |
| 2 | CG | AcII | AA↓CGTT |
| 2 | CG | AcyI | GR↓CGYC |
| 2 | CG | AsuII | TT↓CGAA |
| 2 | CG | ClaI | AT↓CGAT |
| 2 | CG | HinP1I | G↓CGC |
| 2 | CG | HpaII | C↓CGG |
| 2 | CG | MaeII | A↓CGT |
| 2 | CG | NarI | GG↓CGCC |
| 2 | CG | TaqI | T↓CGA |
| 2 | MK | AccI | GT↓MKAC |
| 2 | NN | BceAI | ACGGC (12/14) |
| 2 | NN | BscAI | GCATC (4/6) |
| 2 | NN | BspD6I | GACTC (4/6) |
| 2 | NN | FauI | CCCGC (4/6) |
| 2 | NN | Hpy178III | TC↓NNGA |
| 2 | TA | CviQI | G↓TAC |
| 2 | TA | MaeI | C↓TAG |
| 2 | TA | MseI | T↓TAA |
| 2 | TA | NdeI | CA↓TATG |
| 2 | TA | VspI | AT↓TAAT |
| 3 | ANT | HinfI | G↓ANTC |
| 3 | AWT | TfiI | G↓AWTC |
| 3 | CWG | PasI | CC↓CWGGG |
| 3 | CWG | TseI | G↓CWGC |
| 3 | GNC | AsuI | G↓GNCC |
| 3 | GNC | DraII | RG↓GNCCY |
| 3 | GTC | SimI | GGGTC (−3/0) |
| 3 | GWC | AvaII | G↓GWCC |
| 3 | GWC | PpuMI | RG↓GWCCY |
| 3 | GWC | RsrII | CG↓GWCCG |
| 3 | GWC | SanDI | GG↓GWCCC |
| 3 | GWC | Sse8647I | AG↓GWCCT |
| 3 | NNN | Ksp632I | CTCTTC (1/4) |
| 3 | NNN | SapI | GCTCTTC (1/4) |
| 3 | TCA | BbvCI | CCTCAGC (−5/−2) |
| 3 | TNA | Bpu10I | CCTNAGC (−5/−2) |
| 3 | TNA | DdeI | C↓TNAG |
| 3 | TNA | EspI | GC↓TNAGC |
| 3 | TNA | SauI | CC↓TNAGG |
| 4 | AATT | ApoI | R↓AATTY |
| 4 | AATT | EcoRI | G↓AATTC |
| 4 | AATT | MfeI | C↓AATTG |
| 4 | AATT | TspEI | ↓AATT |
| 4 | ACGA | BsiI | CACGAG (−5/−1) |
| 4 | AGCT | HindIII | A↓AGCTT |
| 4 | CATG | BspHI | T↓CATGA |
| 4 | CATG | BspLU11I | A↓CATGT |
| 4 | CATG | FatI | ↓CATG |
| 4 | CATG | NcoI | C↓CATGG |
| 4 | CCAG | BseYI | CCCAGC (−5/−1) |
| 4 | CCGG | AgeI | A↓CCGGT |
| 4 | CCGG | BetI | W↓CCGGW |
| 4 | CCGG | BspMII | T↓CCGGA |
| 4 | CCGG | Cfr10I | R↓CCGGY |
| 4 | CCGG | Eco56I | G↓CCGGC |
| 4 | CCGG | SgrAI | CR↓CCGGYG |
| 4 | CCGG | Sse232I | CG↓CCGGCG |
| 4 | CCGG | XmaI | C↓CCGGG |
| 4 | CGCG | AscI | GG↓CGCGCC |
| 4 | CGCG | BsePI | G↓CGCGC |
| 4 | CGCG | MauBI | CG↓CGCGCG |
| 4 | CGCG | MluI | A↓CGCGT |
| 4 | CGCG | SeII | ↓CGCG |
| 4 | CNNG | SecI | C↓CNNGG |
| 4 | CRYG | AfIIII | A↓CRYGT |
| 4 | CRYG | DsaI | C↓CRYGG |
| 4 | CTAG | AvrII | C↓CTAGG |
| 4 | CTAG | NheI | G↓CTAGC |
| 4 | CTAG | SpeI | A↓CTAGT |
| 4 | CTAG | XbaI | T↓CTAGA |
| 4 | CWWG | StyI | C↓CWWGG |
| 4 | GATC | BamHI | G↓GATCC |
| 4 | GATC | BcII | T↓GATCA |
| 4 | GATC | BgIII | A↓GATCT |
| 4 | GATC | MboI | ↓GATC |
| 4 | GATC | XhoII | R↓GATCY |
| 4 | GCGC | KasI | G↓GCGCC |
| 4 | GGCC | Bsp120I | G↓GGCCC |
| 4 | GGCC | CfrI | Y↓GGCCR |
| 4 | GGCC | GdiII | CGGCCR (−5/−1) |
| 4 | GGCC | NotI | GC↓GGCCGC |
| 4 | GGCC | XmaIII | C↓GGCCG |
| 4 | GTAC | Asp718I | G↓GTACC |
| 4 | GTAC | Bsp1407I | T↓GTACA |
| 4 | GTAC | SpII | C↓GTACG |
| 4 | GTAC | TatI | W↓GTACW |
| 4 | GYRC | HgiCI | G↓GYRCC |
| 4 | NNNN | AarI | CACCTGC (4/8) |
| 4 | NNNN | AceIII | CAGCTC (7/11) |
| 4 | NNNN | Bbr7I | GAAGAC (7/11) |
| 4 | NNNN | BbvI | GCAGC (8/12) |
| 4 | NNNN | BbvII | GAAGAC (2/6) |
| 4 | NNNN | BsmAI | GTCTC (1/5) |
| 4 | NNNN | BsmFI | GGGAC (10/14) |
| 4 | NNNN | BspMI | ACCTGC (4/8) |
| 4 | NNNN | BtgZI | GCGATG (10/14) |
| 4 | NNNN | Eco31I | GGTCTC (1/5) |
| 4 | NNNN | Esp3I | CGTCTC (1/5) |
| 4 | NNNN | FokI | GGATG (9/13) |
| 4 | NNNN | SfaNI | GCATC (5/9) |
| 4 | NNNN | Sth132I | CCCG (4/8) |
| 4 | NNNN | StsI | GGATG (10/14) |
| 4 | TCGA | AbsI | CC↓TCGAGG |
| 4 | TCGA | PspXI | VC↓TCGAGB |
| 4 | TCGA | SaII | G↓TCGAC |
| 4 | TCGA | SgrDI | CG↓TCGACG |
| 4 | TCGA | XhoI | C↓TCGAG |
| 4 | TGCA | ApaLI | G↓TGCAC |
| 4 | TGCA | Ppu10I | A↓TGCAT |
| 4 | TRYA | SfeI | C↓TRYAG |
| 4 | TTAA | AfIII | C↓TTAAG |
| 4 | TYRA | SmII | C↓TYRAG |
| 4 | YCGR | AvaI | C↓YCGRG |
| 5 | CCNGG | PfoI | T↓CCNGGA |
| 5 | CCNGG | SsoII | ↓CCNGG |
| 5 | CCSGG | EcoHI | ↓CCSGG |
| 5 | CCWGG | EcoRII | ↓CCWGG |
| 5 | CCWGG | SexAI | A↓CCWGGT |
| 5 | GGNCC | UnbI | ↓GGNCC |
| 5 | GGWCC | VpaK11AI | ↓GGWCC |
| 5 | GTNAC | BstEII | G↓GTNACC |
| 5 | GTNAC | MaeIII | ↓GTNAC |
| 5 | GTSAC | Tsp45I | ↓GTSAC |
| 5 | NNNNN | HgaI | GACGC (5/10) |

TABLE 2
Type II restriction enzymes
producing 3′-overhangs

| Length | Overhang | Enzymes | Recognition Sequence |
|---|---|---|---|
| 1 | N | BciVI | GTATCC (6/5) |
| 1 | N | BfiI | ACTGGG (5/4) |
| 1 | N | Eam1105I | GACNNN↓NNGTC |
| 1 | N | Hin4II | CCTTC (6/5) |
| 1 | N | HphI | GGTGA (8/7) |
| 1 | N | Hpy188I | TCN↓GA |
| 1 | N | MboII | GAAGA (8/7) |
| 1 | N | MnII | CCTC (7/6) |
| 1 | N | Tsp4CI | ACN↓GT |
| 1 | N | XcmI | CCANNNNN↓NNNNTGG |
| 1 | S | AgsI | TTS↓AA |
| 2 | AT | BspKT6I | GAT↓C |
| 2 | AT | PacI | TTAAT↓TAA |
| 2 | AT | PvuI | CGAT↓CG |
| 2 | AT | SgfI | GCGAT↓CGC |
| 2 | CG | HhaI | GCG↓C |
| 2 | CN | BsmI | GAATGC (1/−1) |
| 2 | GC | McaTI | GCGC↓GC |
| 2 | GC | SacII | CCGC↓GG |
| 2 | GN | BsrI | ACTGG (1/−1) |
| 2 | NN | ApyPI | ATCGAC (20/18) |
| 2 | NN | AquII | GCCGNAC (20/18) |
| 2 | NN | AquIII | GAGGAG (20/18) |
| 2 | NN | AquIV | GRGGAAG (19/17) |
| 2 | NN | Bce83I | CTTGAG (16/14) |
| 2 | NN | BsbI | CAACAC (21/19) |
| 2 | NN | BseMII | CTCAG (10/8) |
| 2 | NN | BseRI | GAGGAG (10/8) |
| 2 | NN | BsgI | GTGCAG (16/14) |
| 2 | NN | BspCNI | CTCAG (9/7) |
| 2 | NN | BsrDI | GCAATG (2/0) |
| 2 | NN | BstF5I | GGATG (2/0) |
| 2 | NN | BtsI | GCAGTG (2/0) |
| 2 | NN | BtsIMutI | CAGTG (2/0) |
| 2 | NN | CchII | GGARGA (11/9) |
| 2 | NN | CchIII | CCCAAG (20/18) |
| 2 | NN | CdpI | GCGGAG (20/18) |
| 2 | NN | CjeNIII | GKAAYG (19/17) |
| 2 | NN | CstMI | AAGGAG (20/18) |
| 2 | NN | DraRI | CAAGNAC (20/18) |
| 2 | NN | DrdI | GACNNNN↓NNGTC |
| 2 | NN | EciI | GGCGGA (11/9) |
| 2 | NN | Eco57I | CTGAAG (16/14) |
| 2 | NN | Eco57MI | CTGRAG (16/14) |
| 2 | NN | GsuI | CTGGAG (16/14) |
| 2 | NN | HauII | TGGCCANNNNNNNNNNN↓ |
| 2 | NN | MaqI | CRTTGAC (21/19) |
| 2 | NN | MmeI | TCCRAC (20/18) |
| 2 | NN | NlaCI | CATCAC (19/17) |
| 2 | NN | NmeAIII | GCCGAG (21/19) |
| 2 | NN | PlaDI | CATCAG (21/19) |
| 2 | NN | PspOMII | CGCCCAR (20/18) |
| 2 | NN | PspPRI | CCYCAG (15/13) |
| 2 | NN | RceI | CATCGAC (20/18) |
| 2 | NN | RdeGBII | ACCCAG (20/18) |
| 2 | NN | RpaI | GTYGGAG (11/9) |
| 2 | NN | RpaBI | CCCGCAG (20/18) |
| 2 | NN | RpaB5I | CGRGGAC (20/18) |
| 2 | NN | SdeAI | CAGRAG (21/19) |
| 2 | NN | SstE37I | CGAAGAC (20/18) |
| 2 | NN | TagII | GACCGA (11/9) |
| 2 | NN | TsoI | TARCCA (11/9) |
| 2 | NN | TspDTI | ATGAA (11/9) |
| 2 | NN | TspGWI | ACGGA (11/9) |
| 2 | NN | Tth111II | CAARCA (11/9) |
| 2 | NN | WviI | CACRAG (21/19) |
| 2 | RY | McrI | CGRY↓CG |
| 2 | TA | PabI | GTA↓C |
| 3 | CNG | BthCI | GCNG↓C |
| 3 | CSG | TauI | GCSG↓C |
| 3 | GNC | FmuI | GGNC↓C |
| 3 | GNC | PssI | RGGNC↓CY |
| 3 | GWC | Psp03I | GGWC↓C |
| 3 | NNN | AlwNI | CAGNNN↓CTG |
| 3 | NNN | BgII | GCCNNNN↓NGGC |
| 3 | NNN | BsiYI | CCNNNNN↓NNGG |
| 3 | NNN | BstAPI | GCANNNN↓NTGC |
| 3 | NNN | DraIII | CACNNN↓GTG |
| 3 | NNN | MwoI | GCNNNNN↓NNGC |
| 3 | NNN | PflMI | CCANNNN↓NTGG |
| 3 | NNN | RleAI | CCCACA (12/9) |
| 3 | NNN | SfiI | GGCCNNNN↓NGGCC |
| 4 | ACGT | AatII | GACGT↓C |
| 4 | ACGT | TaiI | ACGT↓ |
| 4 | AGCT | SacI | GAGCT↓C |
| 4 | ASST | SetI | ASST↓ |
| 4 | CATG | NlaIII | CATG↓ |
| 4 | CATG | NspI | RCATG↓Y |
| 4 | CATG | SphI | GCATG↓C |
| 4 | CCAG | GsaI | CCCAGC (−1/−5) |
| 4 | CCGG | FseI | GGCCGG↓CC |
| 4 | CTAG | AceII | GCTAG↓C |
| 4 | DGCH | SduI | GDGCH↓C |
| 4 | GATC | ChaI | GATC↓ |
| 4 | GCGC | BbeI | GGCGC↓C |
| 4 | GCGC | HaeII | RGCGC↓Y |
| 4 | GGCC | ApaI | GGGCC↓C |
| 4 | GTAC | KpnI | GGTAC↓C |
| 4 | KGCM | BseSI | GKGCM↓C |
| 4 | NNNN | BstXI | CCANNNNN↓NTGG |
| 4 | RGCY | HgiJII | GRGCY↓C |
| 4 | TGCA | EcoT22I | ATGCA↓T |
| 4 | TGCA | PstI | CTGCA↓G |
| 4 | TGCA | Sse8387I | CCTGCA↓GG |
| 4 | WGCW | HgiAI | GWGCW↓C |
| 4 | YCGR | Nli3877I | CYCGR↓G |
| 5 | CGWCG | Hpy99I | CGWCG↓ |
| 5 | NNNNN | ApaBI | GCANNNNN↓TGC |
| 9 | NNCASTGNN | TspRI | CASTGNN↓ |

The standard IUPAC nucleic acid codes are shown in Table 3 below:

TABLE 3
IUPAC nucleic acid codes

| IUPAC nucleotide code | Base |
|---|---|
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |

In some embodiments, the restriction enzymes do not have a specific recognition sequence.

In some embodiments, the Type II restriction enzyme that generates a single stranded DNA with a 3′-overhang can include but is not limited to BsaXI (Type IIb), BstXI (Type IIp), RleAI (Type IIs) or TstI (Type IIb).

In some embodiments, the Type IIs restriction enzyme that generates a single stranded DNA with a 3′-overhang can include but is not limited to RleAI.

In some embodiments, the Type IIb restriction enzyme that generates a single stranded DNA with a 3′-overhang can include but is not limited to BsaXI.

In some embodiments, the Type IIp restriction enzyme that generates a single stranded DNA with a 3′-overhang can include but is not limited to BstXI.

In some embodiments, the Type IIs restriction enzyme that generates a single stranded DNA with a 5′-overhang can include but is not limited to Earl, BspMI, BsaI, BbsI, or BsmBI.

In some embodiments, the first DNA molecule or the second DNA molecule have single stranded termini generated with an exonuclease.

In some embodiments, the exonuclease that generates single stranded DNA with a 3′-overhang can include but is not limited to T7 exonuclease, T5 exonuclease, or Lambda exonuclease.

In some embodiments, the exonuclease acts on DNA parts that were created via PCR with primers containing phosphorothioate bonds. Primers can also contain other chemically modified bases, such as but not limited to phosphorothioates, locked nucleic acids (LNAs), peptide nucleic acids (PNAs), 2′-0Me nucleotides, methylphosphonates or morpholinos.

In some embodiments, the first DNA molecule or the second DNA molecule have single stranded termini generated with an endonuclease and a second enzyme.

In some embodiments, the endonuclease that generates single stranded DNA with a 3′-overhang can include but is not limited to DNA glycosylase-lyase endonuclease VIII. In some embodiments, the second enzyme used in concert with DNA glycosylase-lyase endonuclease VIII to generate single stranded termini can include but is not limited to uracil DNA glycosylase (UDG).

In some embodiments, the single stranded terminus on one DNA molecule is a 3′-overhang and the single stranded terminus on the other DNA molecule is a 5′-overhang. In some embodiments, the first and second DNA molecules can be ligated using a single stranded DNA (ssDNA) linker (staple).

In some embodiments, the linker is a staple. A staple may be single stranded and can be DNA or RNA. In some embodiments, the staple is a defined sequence capable of binding with perfect complementarity to the single stranded DNA termini generated on the first and second DNA molecules. In some embodiments, the staple binds to a single stranded DNA terminus with a 3′-overhang on a first DNA molecule and a single stranded DNA terminus with a 5′-overhang on a second DNA molecule. In some embodiments, the staple binds to a single stranded DNA terminus with a 5′-overhang on a first DNA molecule and a single stranded DNA terminus with a 3′-overhang on a second DNA molecule.

In some embodiments, the staple is an oligonucleotide between about 4 and about 20 nucleotides in length, and in some embodiments between about 4 nucleotides and about 16 nucleotides, in some embodiments between about 4 nucleotides and about 12 nucleotides, and in some embodiments about 4 nucleotides to about 10 nucleotides in length. In some embodiments, the staple is single stranded DNA or RNA.

In some embodiments, the present invention provides a plurality of reaction mixtures. The reaction mixtures include 1) a first reaction mixture comprising DNA molecules and a restriction enzyme capable of generating a 5′ single stranded DNA terminus for use with the methods of the present invention, 2) a second reaction mixture comprising DNA molecules and a restriction enzyme capable of generating a 3′ single stranded DNA terminus for use with the methods of the present invention, and 3) a third reaction in which the products of the first two reactions are pooled together with a staple linker and ligated. In some embodiments, the first reaction mixture generates a single stranded DNA terminus that is the opposite orientation of the single stranded terminus generated by the second reaction mixture (i.e., one reaction generates a terminus with a 3′-overhang and one reaction generates a terminus with a 5′-overhang). In some embodiments, the single stranded termini generated by both the first and second reaction mixtures are complementary to the staple. In some embodiments, the staple in the reaction mixture contains a defined sequence capable of binding with perfect complementarity to the single stranded terminus generated by the first and second reaction mixtures. In some embodiments, the reaction mixture can contain a staple that is an oligonucleotide between 4 and 10 nucleotides in length, between 4 and 8 nucleotides, or between 6 and 10 nucleotides.

In some embodiments, the first DNA molecule has a single stranded terminus and the second DNA molecule has a single stranded terminus that are each ligated to an intervening double stranded DNA (dsDNA) linker (adapter).

In some embodiments, the linker is an adapter. An adapter is double stranded and can be DNA or RNA. In some embodiments, the adapter contains at least one single stranded terminus containing a degenerate sequence. In some embodiments, the adapter is comprised of oligonucleotides between at least about 5 bp and about 500 bp in length or more, in some embodiments between about 5 bp and about 300 bp, in some embodiments between about 5 bp and about 200 bp and in some embodiments between about 5 bp and 100 bp.

In some embodiments, the single stranded terminus of the adapter is ligated to a 3′ or 5′-overhang of one DNA molecule. In some embodiments, a second single stranded terminus of the adapter is ligated to the 3′ or 5′-overhang of a second DNA molecule. The second single stranded terminus of the adapter can be generated prior to or after ligation of the adapter to the first DNA molecule.

In some embodiments, the present invention provides a plurality of reaction mixtures. The reaction mixtures include 1) a first reaction mixture comprising DNA molecules and enzyme(s) capable of generating a 3′ or 5′ single stranded DNA terminus for use with the methods of the present invention, 2) a second reaction mixture of the same nature as the first reaction but comprising different DNA molecules for use with the methods of the present invention, 3) a third reaction mixture in which the product of the first reaction is ligated to an adapter that contains a degenerate single stranded terminus, 4) a fourth reaction in which the product of the third reaction is pooled with the product of the second reaction and ligated. In some embodiments, the second reaction mixture generates a single stranded DNA terminus that is complementary to the single stranded terminus of the adapter. In some embodiments, the single stranded termini generated by the first reaction mixtures are complementary to the adapter. In some embodiments, the reaction mixture can contain an adapter with a single stranded terminus that contains a degenerate sequence capable of binding to a single stranded DNA terminus complementary to the single stranded DNA terminus generated by the first reaction mixture. In some embodiments, the reaction mixture can contain an adapter that is between 5 and 100 bp in length.

The methods of the present invention can be repeated as tandem steps to assemble final ligation products that contain at least about 2, 3, 4, 5, 6, 7, 8, 9, 10, 11, 12, 13, 14, 15, 20, 30, 40, 50, 70, 100 or more starting molecules, such as DNA or RNA molecules.

The present invention can be employed to assemble, for example plasmids, cosmids, and genomes, of novel sequence. The utility of engineered and synthetic DNA can be found throughout life sciences. In some embodiments, the methods of the present invention generate nucleic acid molecules that are linear or circular. Molecules generated by the methods of the present invention can include but are not limited to plasmids, cosmids, operons, genes, synthetic genes, complete genes, partial genomes, complete genomes, partial synthetic genomes, and complete synthetic genomes. Molecules generated by the methods of the present invention can also include naturally occurring pathway components or synthetically derived pathway components.

In some embodiments, the assembly of the desired nucleic acid molecule can be performed in a single step. In some embodiments, the step is a single isothermal step. According to the present methods, the nucleic acid portions of the invention desired to be assembled are combined with appropriate staples and an assembly buffer to form a reaction mixture. The assembly buffer can include for example, the desired restriction and ligase enzymes necessary to assemble the nucleic acid. In some embodiments, the assembly buffer includes restriction enzymes (at least one 5′-overhang-generating enzyme and at least one 3′-overhang-generating enzyme) and DNA ligase (e.g., T7 DNA ligase). The reaction mixture can then be incubated at a single temperature reaction (i.e., isothermal reaction) that allows for digestion, annealing and ligation steps. In some embodiments the temperature is about 30° C. to about 50° C., about 30° C. to about 40° C., about 37° C. to about 42° C., about 37° C. or about 42° C. In some embodiments, the reaction mixture is incubated at 37° C. and all necessary digestion, annealing and ligation steps occur to assemble DNA and/or RNA molecules together. In some embodiments, at least about 2 to 100 or more DNA and/or RNA molecules are assembled in a isothermal reaction. In some embodiments at least about 2 to about 100, about 2 to 70, about 2 to 50, about 2 to 20, about 2 to about 12, about 2 to about 10, about 2 to about 8, about 2 to 6, about 2 to 4 or about 2 DNA and/or RNA molecules are assembled in an isothermal reaction. In some embodiments, at least about 2, 3, 4, 5, 6, 7, 8, 9, 10, 11, 12, 13, 14, 15, 20, 30, 40, 50, 70, 100 DNA and/or RNA molecules are assembled in an isothermal reaction.

The methods of the present invention further provide for the ability to multiplex different assemblies within the same reaction vessel. Multiple reactions can be carried out in the same buffer due to the specificity afforded by each staple. As a result, assembly reagents can be minimized while increasing the productivity of an assembly process.

In some embodiments, the DNA molecules generated by the present invention can be transformed or transfected into a variety of cells, including but not limited to bacteria, insect and mammal cells. The DNA molecules of the present invention can also be inserted into viruses or virus-like particles. Transfection and transformation methods are well known in the art and any standard methods can be employed with the present invention.

The selection of nucleic acid parts, restriction enzymes, and staples, as well as the individual reaction assemblies and/or reagents employed therein, can be determined computationally, taking into account a variety of parameters, including logistical, cost and biophysical parameters. For example, the reaction assemblies and assembly routes can be guided by limitations or parameters for enzymes or other reagents, as experimentally-derived or known from the literature, and/or guided by cost, availability, or compatibility of the various reagents.

Parameters can include logistical parameters. In some embodiments, logistical parameters for designing the assembly route include logistical considerations such as part availability or historical performance metrics. Part availability can include availability of nucleic acid sequences, restriction enzymes, buffers, or any other reagent employed with the multipart, modular and scarless assembly described herein. Historical performance can include but is not limited to compatibility of reagents, efficiency of reagents, and/or specificity of reagents.

Parameters can include financial parameters. In some embodiments, financial parameters may address part cost, manipulation, reagents, and/or overhead. Consideration of financial parameters may determine that certain optimal parts should be synthesized by de novo nucleic acid synthesis (rather than scarless assembly).

Parameters can also include functional or biophysical parameters. Ligation conditions and/or enzymatic digestion conditions are exemplary functional parameters. In some embodiments, an algorithm selects nucleic acid parts based on desired functional properties of the desired sequence. For instance, the algorithm can select DNA parts that encode promoters, ribosome binding sites, terminators, or other regulatory elements to elicit designed levels of gene expression.

In some embodiments, the method utilizes an algorithm to determine and/or optimize the steps for assembling a complex nucleic acid molecule, i.e., for assembly of a multipart, modular and scarless nucleic acid sequence. In some embodiments, the algorithm selects reaction reagents to ensure sufficient reaction efficiency and fidelity during multiplex reactions and across multiple rounds of nucleic acid assembly. Reaction efficiency and fidelity can be predicted from empirical and biophysical data, and can include selecting the number and composition of nucleic acid parts in each reaction. For example, empirical data might suggest a maximum of 5 nucleic acid parts per reaction based on ligation efficiency. In some exemplary embodiments, the algorithm would determine that a 10 part nucleic acid assembly be split into 3 reactions spanning 2 iterative rounds of assembly to produce the final nucleic acid molecule.

In some embodiments, ssDNA overhangs generated during assembly must be specific to ensure correct assembly. In some embodiments, the algorithm identifies incompatible ssDNA overhangs and separates component parts into different reactions in order to ensure specificity of assembly.

In some embodiments, the algorithm considers specifications and limitations of automation hardware when determining the required and/or optimal assembly steps. Such specifications and/or limitations can include, for example, but are not limited to volume tolerances of a liquid handling robot, speed of execution, and throughput of the system.

The present invention also provides for kits. Kits contemplated by the methods of the of the present invention can include 1) a single stranded staple or a double stranded terminus adapter, 2) enzymes capable of generating single stranded DNA termini and 3) an instruction for use. In some embodiments, the kit comprises a DNA ligase, a 5′-overhang-generating enzyme, and a 3′-overhang-generating enzyme. In some embodiments, the kit comprises the enzymes capable of generating single stranded DNA termini and an appropriate buffer for enzyme function. In some embodiments, the kit comprises a standard set of staples. In some embodiments, the staples are not part of the kit. In some embodiments, the kit comprise a plurality of reaction mixtures. In some embodiments, the kit comprises a plurality of adapters and enzymes for performing a plurality of reactions.

In some embodiments, the kit further comprises an implementation of an algorithm as described herein, i.e. software for use according to the present methods.

In some embodiments, the enzyme in the kit for generating the 5′-overhang is selected from Type IIs, Type IIb or Type IIp restriction enzymes or combinations thereof, including those listed in Table 1. In some embodiments, the enzymes in the kit for generating the 5′-overhang is selected from EarI, BspMI, BsaI, BbsI, and BsmBI, or combinations thereof. In some embodiments, the enzyme in the kit for generating the 3′-overhang is selected from Type IIs, Type IIb or Type IIp restriction enzymes or combinations thereof, including those listed in Table 2. In some embodiments, the enzymes in the kit for generating the 3′-overhang is selected from BsaXI, RleAI, and TstI and combinations thereof.

### EXAMPLES

### Example 1

### Staple Method

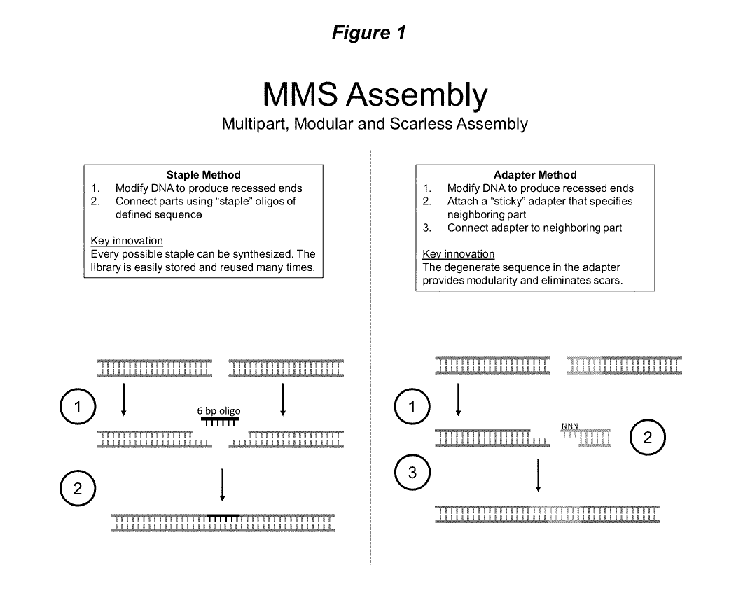

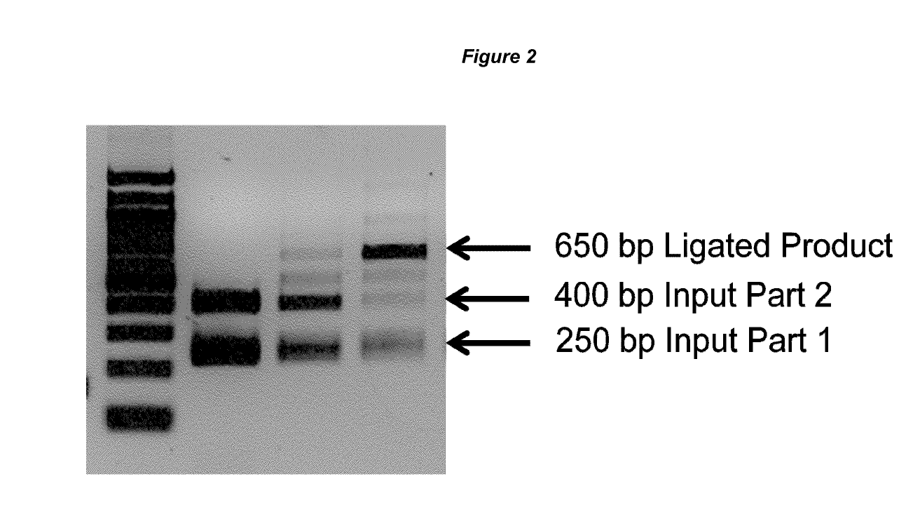

One example of the methods is the “Staple Method.” DNA parts are prepared by digestion with Type IIs restriction enzymes to generate termini with 5′ and 3′ single stranded DNA overhangs. Most Type IIs enzymes create short single stranded DNA overhangs (about 2 bp to 6 bp). This results in a relatively small “gap” at the junction between two DNA parts. This “gap” can be filled by a defined oligonucleotide (i.e., staple linker) that is perfectly complementary to the generated single stranded DNA overhangs. The oligonucleotide spans the junction and anneals to both the 5′ single stranded DNA overhang of one part and the 3′ single stranded DNA overhang of the other part. More than two DNA parts can be simultaneously joined together, and the order of assembly will be dictated by the sequence of the oligonucleotides provided in the reaction. See, for example, FIGS. 1 and 2.

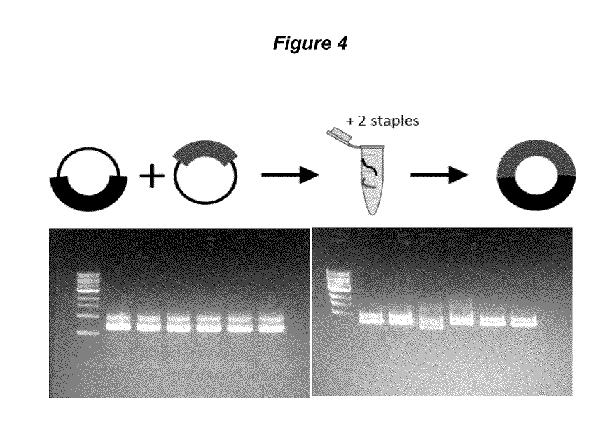

The staple method can also be employed in performing isothermal scarless subcloning. For isothermal scarless subcloning, the reaction mixture contained T4 ligase buffer, T7 DNA ligase, BsaI and BsaXI, and DNA parts. The isothermal reaction was performed at 37° C. for 1 hr. Colony PCRs and sequencing show that 11 of 12 clones assembled correctly. See, for example, FIG. 4.

In another example, the reaction mixture contained T4 ligase buffer, T7 DNA ligase, BsaI and BstXI, and DNA parts. The isothermal reaction was performed at 37° C. for 8 hr. Colony PCRs and sequencing show that 6 of 12 clones assembled correctly.

In a further example, performing the multiplex assembly in one tube, the reaction mixture contained T4 ligase buffer, T7 DNA ligase, BsaI and BstXI, and DNA parts. The isothermal reaction was performed at 37° C. for 8 hr. Colony PCRs and sequencing show that 23 of 24 clones assembled correctly.

### Example 2

### Adapter Method

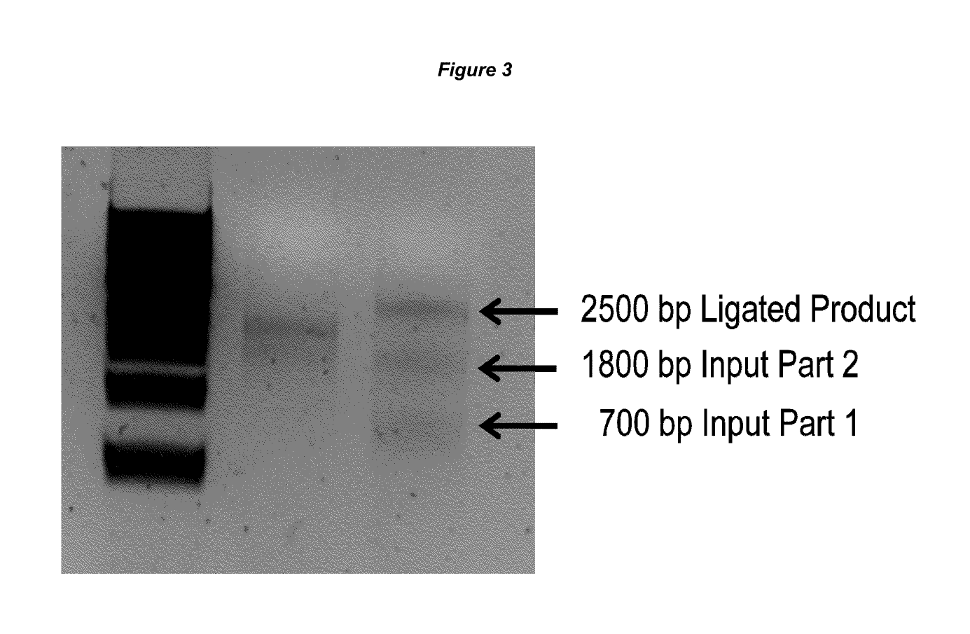

A second example of the methods is the “Adapter Method.” A dsDNA adapter (i.e., single stranded terminus adapter) is created for each part (linker paired part or LPP) such that it contains a single stranded DNA termini comprising degenerate bases, e.g. NNNN. The dsDNA sequence in the adapter can either duplicate the terminal sequence of the LPP, or it can serve as a replacement for the terminal sequence of the LPP. In the latter case, the LPP would be reconstructed to be a smaller size. In the “Adapter Method,” DNA parts are modified with restriction enzymes to generate single stranded DNA termini. The adapter corresponding to the desired neighboring part is then ligated to the single stranded DNA termini. Finally, the adapter is joined to its LPP. In the accompanying example, we utilized the second class of assembly (exonuclease based) to ligate the adapter to its LPP. See, for example, FIGS. 1 and 3.

It is understood that the examples and embodiments described herein are for illustrative purposes only and that various modifications or changes in light thereof will be suggested to persons skilled in the art and are to be included within the spirit and purview of this application and scope of the appended claims. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims. Those skilled in the art will recognize, or be able to ascertain using no more than routine experimentation, many equivalents to the specific embodiments of the invention described herein. Such equivalents are intended to be encompassed by the appended claims.

All publications, patents, and patent applications cited herein are hereby incorporated by reference in their entirety for all purposes.

1. A method for scarless assembly of two or more DNA molecules, said method comprising:
generating a first DNA molecule having a single stranded terminus,
generating a second DNA molecule having a single stranded terminus,
ligating the first and second DNA molecules such that the ligation product corresponds to the combined sequence of the first and second DNA molecules.

2. The method of claim 1, wherein the following reactions are performed:
a. generating a first DNA molecule having a 5′ single stranded overhang;
b. generating a second DNA molecule having a 3′ single stranded overhang;
c. providing a short oligonucleotide staple linker containing perfect or near perfect complementarity to the 5′ and 3′-overhangs; and
d. ligating the first DNA molecule, the second DNA molecule, and the staple linker.

3. The method of any of the preceding claims, wherein the 5′ or 3′ single stranded overhangs are generated with a restriction enzyme.

4. The method of any of the preceding claims, wherein a Type IIs restriction enzyme generates DNA with 3′ single stranded overhangs.

5. The method of any of the preceding claims, wherein a Type IIb restriction enzyme generates DNA with 3′ single stranded overhangs.

6. The method of any of the preceding claims, wherein a Type IIp restriction enzyme generates DNA with 3′ single stranded overhangs.

7. The method of any of the preceding claims, wherein a Type IIs restriction enzyme generates DNA with 3′ single stranded overhangs, and the Type IIs restriction enzyme is optionally RleAI.

8. The method of any of the preceding claims, wherein a Type IIb restriction enzyme generates DNA with 3′ single stranded overhangs, and the Type IIb restriction enzyme is optionally BsaXI.

9. The method of any of the preceding claims, wherein a Type IIp restriction enzyme generates DNA with 3′ single stranded overhangs, and the Type IIp restriction enzyme is optionally BstXI.

10. The method of any of the preceding claims, wherein the Type IIs restriction enzyme generates DNA with 5′ single stranded overhangs.

11. The method of any of the preceding claims, wherein the Type IIs restriction enzyme generates DNA with 5′ single stranded overhangs, and the Type IIs restriction enzyme is optionally selected from EarI, BspMI, BsaI, BbsI, or BsmBI.

12. The method of any of the preceding claims, wherein the single stranded DNA terminus with a 3′ overhang is generated through the action of an exonuclease.

13. The method of any of the preceding claims, wherein the exonuclease digests DNA that was produced by PCR using oligos containing phosphorothioate bonds.

14. The method of any of the preceding claims, wherein the exonuclease is selected from T7 exonuclease, T5 exonuclease, or Lambda exonuclease.

15. The method of any of the preceding claims, wherein the single stranded DNA terminus with a 3′-overhang is generated through the action of uracil DNA glycosylase (UDG) and DNA glycosylase-lyase endonuclease VIII.

16. The method of any of the preceding claims, wherein the staple linker contains a defined sequence capable of binding with perfect or near perfect complementarity to the single stranded DNA termini of the first and second DNA molecules.

17. The method of any of the preceding claims, wherein the staple linker binds to both a single stranded terminus with a 3′-overhang and a single stranded terminus with a 5′-overhang.

18. The method of any of the preceding claims, wherein the single stranded terminus with a 3′-overhang and the single stranded terminus with a 5′-overhang are ligated together with the staple linker by a DNA ligase, and the DNA ligase enzyme is optionally selected from T4 DNA ligase, T7 DNA ligase, and Taq DNA ligase.

19. The method of any of the preceding claims, wherein the staple linker is an oligonucleotide of DNA, RNA, or modified DNA and RNA molecules between 4 and 20 nucleotides in length.

20. The method of any of the preceding claims, wherein the staple linker contains single stranded DNA, double stranded DNA, or combination thereof.

21. The method of any of the preceding claims, wherein the ligating step involves a single stranded terminus adapter containing a degenerate sequence or a defined sequence.

22. The method of any of the preceding claims, wherein the single stranded terminus adapter contains dsDNA.

23. The method of any of the preceding claims, wherein the single stranded terminus adapter is between 5 and 100 nucleotides in length.

24. The method of any of the preceding claims, wherein the single stranded terminus adapter duplicates the terminal sequence of the second DNA molecule.

25. The method of any of the preceding claims, wherein the single stranded terminus adapter includes a single stranded DNA terminus of defined sequence.

26. The method of any of the preceding claims, wherein the single stranded terminus adapter and/or the second DNA molecule are modified via the action of an exonuclease.

27. The method of any of the preceding claims, wherein the single stranded terminus adapter contains a degenerate sequence capable of binding to a single stranded DNA terminus complementary to the single stranded DNA terminus.

28. The method of any of the preceding claims, wherein the single stranded terminus adapter is between 5 and 100 nucleotides in length.

29. The method of any of the preceding claims, wherein the single stranded DNA terminus of the single stranded terminus adapter and second DNA molecule are annealed and ligated.

30. A reaction mixture capable of generating a 3′ single stranded DNA terminus overhang according to the method of any of the preceding claims.

31. A reaction mixture capable of generating a 5′ single stranded DNA terminus overhang according to the method of any of the preceding claims.

32. A reaction mixture comprising enzymes capable of generating 3′, 5′, and/or combination of 3′ and 5′ single stranded DNA terminus overhang in a single reaction, according to the method of any of the preceding claims.

33. The reaction mixture of any of the preceding claims, wherein the restriction enzyme is a Type IIs, Type IIb or Type IIp restriction enzyme.

34. The reaction mixture of any of the preceding claims, wherein the Type IIs, Type IIb or Type IIp restriction enzyme is selected from BsaXI, RleAI, and TstI and the restriction enzyme generates single stranded terminus with a 3′-overhang.

35. The reaction mixture of any of the preceding claims, wherein the Type IIs restriction enzyme is selected from EarI, BspMI, BsaI, BbsI, and BsmBI and the restriction enzyme generates single stranded terminus with a 5′-overhang.

36. A reaction mixture for performing the method of any of the preceding claims.

37. The method of any of the preceding claims, in which the product of scarless assembly method is circular DNA.

38. The method any of the preceding claims, in which the product of scarless assembly method can be transformed or transfected into cells.

39. The method or reaction mixture of any of the preceding claims, wherein more than two DNA molecules are simultaneously ligated together.
